(19) United States
(12) Patent Application Publication
(10) Pub. No.: US 2025/0191757 A1
(43) Pub. Date: Jun. 12, 2025

(54) A METHOD OF PRECISION TREATMENT

(71) Applicant: POLYGENRX PTY LTD, Waratah, New South Wales (AU)

(72) Inventors: Murray CAIRNS, Waratah, New South Wales (AU); William REAY

(73) Assignee: POLYGENRX PTY LTD, Waratah, New South Wales (AU)

(21) Appl. No.: 18/844,780

(22) PCT Filed: Apr. 21, 2023
(86) PCT No.: PCT/AU2023/050326

§ 371 Date: Sep. 6, 2024

(30) Foreign Application Priority Data
Mar. 8, 2022 (AU) 2022900558

(51) Int. Cl. G16H 50/20; G16B 20/20; G16B 50/10; G16H 10/60; G16H 20/10; G16H 50/70

(52) U.S. Cl. CPC G16H 50/20; G16B 20/20; G16B 50/10; G16H 10/60; G16H 20/10; G16H 50/70

(57) ABSTRACT

Disclosed herein are methods for the precision treatment of human subjects with a complex disorder or disorders, comprising identifying directional anchors that constitute either therapeutic agents or therapeutic targets to treat the disorder and quantifying common variant enrichment amongst genes biologically annotated as linked to the directional anchor. As described herein, the quantification of common variant enrichment amongst the genes biologically linked to a directional anchor provides a means of summating an individual's exposure burden to genetic risk variants that is potentially treatable by a pharmacological agent or intervention that is related to a directional anchor. In other words, the quantified burden of genetic risk amongst genes linked to a directional anchor may inform individuals more sensitive to treatment by that intervention or an intervention which targets it.

### FIELD

This disclosure relates generally to methods for treating complex diseases in a human subject. Specifically, this comprises constructing a genetic risk score orientated around genes related to the mode of action of a specific agent, and thus, selecting a suitable agent for treatment in an individual through the use of this genetic score.

### BACKGROUND

The vast majority of global disease burden is underpinned by complex disorders, including, but not limited to, psychiatric and neurobehavioural disorders, neurodegenerative disorders, inflammatory and autoimmune disorders, metabolic and cardiovascular disease, cancer, and renal disease. Genetic risk plays an intrinsic role in common human disease and provides insights to may assist to improve patient outcomes. Although, genome-wide association studies (GWAS) have revealed much of the complexity of the heritable component of these traits, we will need innovative approaches to translate vast amounts of genetic data available into clinically actionable insights.

A key aspect of genetic component of complex disorders is that inter-individual heterogeneity is pervasive. In other words, the precise genetic risk factors carried by any given patient will be highly variable, and this can result in similarly variable biology being impacted by the genetic architecture of a disorder. Understanding these differences between individuals is likely crucial to facilitate precision management of these disorders and assist in treatment formulation. Conventional approaches that summate genetic risk burden in an individual, such as a polygenic risk scoring (PRS), also commonly referred to as polygenic scoring (PGS), do so by weighting individual alleles carried genome-wide by their association effect size from a well powered GWAS for the trait or disorder in question. These PRS/PGS approaches have demonstrated significant associations with a diverse range of phenotypes at the population level; for example, heart disease, breast cancer, type 2 diabetes, and inflammatory bowel disease (Khera et al., 2018, Nature Genetics, 50: 1219-1224).

Whilst genome wide PRS/PGS can model individual differences in genetic risk, a key limitation of these methods are their composition of heterogeneous genetic risk factors that lack biological salience and cannot provide specific information that would assist to formulate treatment for a complex disorder. As a result, there is an ongoing need for methodology that utilise the genetic architecture of complex disorders revealed by GWAS in a manner that is informative for treatment. Specialised genetic risk scores, termed pharmagenic enrichment scores (PES), are specifically oriented around clinically actionable, that is, targetable by drugs, biological pathways (Reay et al., 2020, Scientific Reports, 10(1):879). The use of this PES approach is designed around identifying targetable pathways for a disorder with no a priori hypothesis, however, there is no inherent information as to which direction of therapeutic modulation is appropriate.

### SUMMARY

This disclosure is predicated on the application of biologically directed polygenic scores, that is, apharmagenic enrichment score, directed to genes associated with either a specific therapeutic agent or a specific therapeutic target, whereby the direction of beneficial therapeutic modulation can be predicted genetically. A therapeutic agent in this context includes, but is not limited to, a pharmacological agent, lifestyle intervention, or non-prescription supplement; moreover, a therapeutic target encompasses a gene and its associated mRNA, mRNA isoforms thereof, protein, protein isoforms thereof, or post-translational modifications of said protein. Both of the above, that is, a therapeutic agent or therapeutic target, are hereafter referred to collectively as a directional anchor.

Accordingly, the present disclosure provides a mechanism for treating a complex disorder in a human subject comprising:
a. selecting a suitable directional anchor in the form of a therapeutic agent or therapeutic target around which precision treatment of a complex disorder in a human subject could be implemented;
b. selecting a directional anchor comprises the following steps for a therapeutic agent;
   i. identify a therapeutic agent for which a target gene, or genes, can be reasonably inferred, whereby a target refers to a gene modified in some fashion by the agent;
c. selecting a directional anchor comprises the following steps for a therapeutic target;
   i. identify a gene, or genes, whereby the direction of expression, encompassing mRNA or protein expression, associated with increased risk of the complex disorder can be proposed or predicted;
d. identifying genes related to the directional anchor from step b or c are identified from a plurality of data sources, including but not limited to, predicted proteins that interact with said gene/s, genes linked to said gene/s via evidence amassed in scientific literature, or genes correlated with an experiment whereby the effect of a therapeutic agent selected via the process outlined in step b is examined;
e. obtaining data representing genome-wide variant effect sizes from a plurality of individuals with the complex disorder and a plurality of individuals without the complex disorder;
f. selecting a plurality of variants physically mapped to genes, or proximal thereof, from step d and weighting them by their effect size from the genome-wide variant effect sizes;
g. treating a subject with the therapeutic agent guided by the polygenic score, whereby the therapeutic agent is selected from step b or an agent targeting factors selected using the process in step c, this comprises;
   i. calculating a pharmagenic enrichment score by summating the variant effect sizes from step f,
   ii. identifying whether the individual will be sensitive to treatment with this agent based on whether the numeric value of the pharmagenic enrichment score is elevated relative to a reference population for which that score is also calculated.

The method of treatment described above represents a key advance in that the application of the pharmagenic enrichment score is specifically orientated around a selected therapeutic, and thus, the direction of effect of the selected therapeutic is known or predicted.

### BRIEF DESCRIPTION OF THE DRAWINGS

Embodiments of the disclosure are described herein, by way on non-limiting example, with reference to the accompanying drawings.

FIG. 1 is a schematic representation of a directional anchor in the form of a therapeutic target. For instance, if increased expression of a gene is associated with a disorder through a transcriptome or proteome-wide association study (TWAS/PWAS) or Mendelian randomisation (MR) using quantitative trait loci as instrumental variables, then an antagonist of said gene may be a repurposing opportunity. Directional anchor genes then act as seed genes to define a network of other genes that interact with them. SNPs mapped to this network are then utilised to construct a pharmagenic enrichment score (PES) for the network. In the case of a binary disease phenotype, the interpretation of the PES would be that individuals with an elevated score relative to an appropriate population reference may benefit from a compound which modulates the directional anchor gene.

FIG. 2 denotes the process of the identification of candidate directional anchors embodied as therapeutic targets and examples thereof for schizophrenia and bipolar disorder. Specifically, panel A is a schematic for the prioritisation of candidate directional anchor genes through models of genetically regulated expression (GReX) and Mendelian randomisation. In both instances, approved compounds are derived for implicated genes that reverse the odds increasing direction of mRNA or protein expression. TWAS=transcriptome-wide association study, PWAS=proteome-wide association study. Panel B displays the results of the multi-tissue (brain and blood) TWAS for schizophrenia (SZ, top) and bipolar disorder (BIP, bottom). The Miami plot visualises the −log 10 transformed P value of association with genes exhibiting a negative genetic covariance between expression and the trait, that is, TWAS Z<0, plotted in the downward direction. The red line denotes the Bonferroni threshold. The candidate directional anchor genes from the TWAS approach are highlighted on the plot along with their putative repurposing candidate the corrects the odds-increasing direction of expression. For example, predicted PCCB expression is negatively correlated with SZ, and thus, a PCCB agonist like biotin may be clinically useful.

FIG. 3 displays biological networks interacting with candidate directional anchors for schizophrenia and bipolar. In panel A, visualisation of two networks of genes that putatively interact with CACNA1C (left) and FADS1 (right) based on experimental and curated database evidence. Blue edges represent evidence from curated databases, whilst purple edges denote experimentally determined evidence. In panel B, Gene-set association (MAGMA) of the entire network for each candidate directional anchor (DA) gene, with and without the DA gene included from the model. Dotted line represents nominal significance (P<0.05). The MAGMA P value is derived from a model which tests whether the common variant signal within genes in the network is greater than what is observed amongst all remaining genes. Two genic boundaries were utilised to annotate SNPs to genes from the GWAS: conservative (5kb upstream, 1.5 kb downstream, left panel) and liberal (35 kb upstream, 10 kb downstream, right panel). In panel C, Kernel density estimation plots of the MAGMA gene-set association P value for each gene-set tested using either schizophrenia or bipolar results, whichever was more significant, which had a significant overrepresentation of genes within that network. The dotted line represents Bonferroni significance for the approximately 34,000 gene-sets considered in the full analysis of all gene-sets that were tested for overrepresentation.

FIG. 4 denotes the schizophrenia and bipolar disorder GRIN2A directional anchor gene network pharmagenic enrichment scores and their relationship with PRS The scatter plots denote the concordance between the scaled unadjusted (raw) GRIN2A network PES for SZ (A) and BIP (B) and both a residualised score and genome wide PRS. Specifically, the left-most scatterplots visualise the relationship between the raw GRIN2A network PES and the residuals from a model which regressed genotyping batch, ten SNP derived principal components, and genome wide PRS for the disorder in question (Residualised GRIN2A PES). The dotted lines represent the 90^{th} percentile of the raw PES and residualised PES, respectively. The points coloured orange (SZ) and red (BIP) indicate individuals with a PES in the 90^{th} percentile or above for both the raw and residualised scores. The right scatterplots plot the relationship between genome wide PRS for SZ or BIP and the GRIN2A network PES. In these instances, the dotted vertical line denotes the 90^{th} percentile of the GRIN2A PES, whilst the horizontal dotted line denotes the 10^{th} percentile of genome wide PRS. As a result, the points coloured purple and blue in the SZ and BIP plots, respectively, are individuals with low relative genome wide PRS (lowest decile) but high GRIN2A PES (highest decile).

FIG. 5 plots the results of the phenome-wide association studies (pheWAS) of each network PES or PRS related to serum or urine biochemical measures and mental health disorders. Heatmap of the association between each network PES and PRS with each trait tested for the biochemical measures (top) and self-reported mental health disorders (bottom). Traits ordering derived from clustering by Pearson's distance. The variable visualised in the heatmaps for the continuous biochemical traits was the regression t value (beta/SE), whilst for the binary mental health phenotypes it was the corresponding Z value from the logistic regression, whereby Z>0 equates to an odds ratio for the disorder>0. Asterisks were utilised to denote the significance of the association−*=P<0.05, **=false discovery rate (FDR)>0.05, and ***=family-wise error rate (FWER)<0.05.

FIG. 6 plots the putative expression derived target genes of the directional anchor as embodied by a therapeutic agent. Volcano plots demonstrate mRNA transcripts which are upregulated and downregulated upon treatment with each of the three compounds, relative to a matched control. The bottom right panel denotes semantic clustering of ontological terms for which genes linked to the directional anchor are overrepresented.

FIG. 7 denotes the statistical effect size of the association of the pharmagenic enrichment score related to the directional anchor embodied by the therapeutic agent of FTO inhibitors. The forest plots indicate the estimated odds ratios of prevalent breast cancer and corresponding 95% confidence intervals for each standard deviation increase each of the configurations of the pharmagenic enrichment score related to these directional anchors tested. The top panel denotes effect sizes unadjusted for background breast cancer genome wide genetic common variant germline risk (PRS), whilst the bottom panel covaries for this metric.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an”, and “the” additionally include their plural aspects unless in the event that their context clearly states otherwise. Therefore, reference to “an agent” includes a single agent, as well as two or more agents, and so on, and so forth.

In its central aspect, the present disclosure provides a method for treating a complex disorder whereby it comprises;
a. selecting a suitable directional anchor in the form of a therapeutic agent or therapeutic target around which precision treatment of a complex disorder in a human subject could be implemented;
b. selecting a directional anchor comprises the following steps for a therapeutic agent;
   i. identify a therapeutic agent for which a target gene, or genes, can be reasonably inferred, whereby a target refers to a gene or genes modified in some fashion by the agent;
c. selecting a directional anchor comprises the following steps for a therapeutic target;
   i. identify a gene, or genes, whereby the direction of expression, encompassing mRNA or protein expression, associated with increased risk of the complex disorder can be proposed or predicted;
d. identifying genes related to the directional anchor from step b or step c are identified from a plurality of data sources, including but not limited to, predicted proteins that interact with said gene/s, genes linked to said gene/s via evidence amassed in scientific literature, or genes correlated with an experiment whereby the effect of a therapeutic agent selected via the process outlined in b is examined;
e. obtaining data representing genome-wide variant effect sizes from a plurality of individuals with the complex disorder and a plurality of individuals without the complex disorder;
f. selecting a plurality of variants physically mapped to genes, or proximal thereof, from step d and weighting them by their effect size from the genome-wide variant effect sizes;
g. treating a subject with the therapeutic agent guided by the polygenic score, whereby the therapeutic agent is selected from step b or an agent targeting factors selected using the process in step c, this comprises;
   i. calculating a pharmagenic enrichment score by summating the variant effect sizes from step f;
   ii. identifying whether the individual will be sensitive to treatment with this agent based on whether the numeric value of the pharmagenic enrichment score is elevated relative to a reference population for which that score is calculated.

The proceeding sections will further delineate specific terminology and components of the method for treatment described above.

The term “Complex Disorders” as used herein refers to disorders which do not display typical patterns of Mendelian inheritance in the majority of instances, that is, they do not arise from a single gene or small set of genes. Moreover, complex disorders result from a complex interplay between heritable (genetic) and environmental components. Complex disorders would be known to those skilled in the art, with some examples for illustration including heart disease, schizophrenia, breast cancer, Parkinson's disease, bipolar disorder, diabetes, asthma, and Crohn's disease.

A complex disorder may also encompass one or many “complex traits”, which is often used interchangeably by those skilled in the art with the term “quantitative trait.” These complex traits also do not exhibit Mendelian inheritance patterns, and exist as a distribution of continuous variables amongst individuals—examples thereof include, height, body-mass index, white blood cell count, high-density lipoprotein, blood pressure, and creatinine.

The term “variant” as used herein refers to any modification to the DNA sequence as compared to one or more reference DNA sequences. Variants may involve any number of adjacent or spaced apart bases or series of bases, and may include single nucleotide substitutions, insertions, deletions, and block substitutions of nucleotides, structural variants, fusion, copy number variants, repeat length variants, variable number tandem repeats, microsatilites, minisatelites.

In an embodiment, variants are selected from the group consisting of common SNPs, CNV, gene deletions, gene inversions, gene duplications, splice variants and haplotypes associated with the complex disorder. In a preferred embodiment, the variants are SNPs.

The term “genome-wide variants” as used herein refers to information pertaining to genetic variants across the whole genome. Such information includes variants in both coding and non-coding regions of the genome.

In an embodiment, the data representing genome-wide variants is selected from the group consisting of single nucleotide polymorphism (SNP) genotype data, copy number variant (CNV) data, gene deletion data, gene inversion data, gene duplication data, splice variant data, haplotype data, or combinations thereof.

In an embodiment, the data representing genome-wide variants is SNP genotype data.

As used herein, the term “SNP” or “single nucleotide polymorphism” refers to a genetic variation between individuals; e.g., a single nitrogenous base position in the DNA of organisms that is variable. As used herein, “SNPs” is the plural of SNP.

The term “polymorphism” as used herein refers to a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

The term “gene” as used herein refers to one or more sequence(s) of nucleotides in a genome that together encode one or more expressed molecules, e.g., an RNA, or polypeptide. The gene can include coding sequences that are transcribed into RNA, which may then be translated into a polypeptide sequence, and can include associated structural or regulatory sequences that aid in replication or expression of the gene.

The term “genotype” as used herein refers to the genetic constitution of an individual (or group of individuals) at one or more genetic loci. Genotype is defined by the allele(s) of one or more known loci of the individual, typically, the compilation of alleles inherited from its parents.

The term “haplotype” as used herein refers to the genotype of an individual at a plurality of genetic loci on a single DNA strand. Typically, the genetic loci described by a haplotype are physically and genetically linked, i.e., on the same chromosome strand.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a SNP, which is a polymorphism at a single nucleotide position in a genome (the nucleotide at the specified position varies between individuals or populations).

The term “allele frequency” as used herein refers to the frequency (proportion or percentage) at which an allele is present at a locus within an individual, within a line, or within a population of lines. For example, for an allele “A” diploid individuals of genotype “AA”, “Aa” or “aa” may have allele frequencies of 2, 1, or 0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “locus” as used herein refers to a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

Methods for obtaining data representing genome-wide variants would be known to persons skilled in the art, illustrative examples of which include performing microarray analysis, massively parallel sequencing, amplicon sequencing, multiplexed PCR, molecular inversion probe assay, GoldenGate assay, allele-specific hybridization, DNA-polymerase-assisted genotyping, ligase-assisted genotyping, and comparative genomic hybridization (CGH). Alternatively, data representing genome-wide variants may be obtained from published datasets.

In an embodiment, the data representing genome-wide variants is obtained from genome-wide association study (GWAS) summary statistics.

It is contemplated herein that the data representing genome-wide variants from the plurality of individuals with the complex disorder and the plurality of individuals without the complex disorder may be obtained using one method, which may differ from the method for obtaining data representing genome-wide variants from the subject. For example, SNP genotype data from the plurality of individuals with the complex disorder and the plurality of individuals without the complex disorder may be obtained by SNP microarray, while the SNP genotype from the subject may be obtained by massively parallel sequencing.

In an embodiment, the data representing genome-wide variants from a plurality of individuals with the complex disorder and a plurality of individuals without the complex disorder is obtained from a GWAS. GWAS are observational studies of a genome-wide set of genetic variants in different individuals to see if any variant is associated with a trait. GWAS have identified a large number of genetic variants significantly associated with human disease. These disease-associated variants have provided candidate genes for further study and hypotheses about disease mechanisms. GWAS have also confirmed the polygenic nature of complex disorders, particularly for psychiatric disorders. For example, GWAS studies have demonstrated that the cumulative effect of a large number of weakly associated SNPs, most of which are not statistically significant alone.

The term effect size would be understood by those skilled in the art as an output from a generalised linear model which represents the effect of a variant, per allele under an additive model, on the phenotype or complex disorder of interest. In an embodiment, these effect sizes represent mean genotype-disorder effects.

A directional anchor hereby refers to a specific biological factor to which the method of treatment is guided. In one embodiment, this is a therapeutic agent around which precision treatment of a complex disorder in a human subject could be implemented. A therapeutic agent in this context includes, but is not limited to, a pharmacological agent, lifestyle intervention, or non-prescription supplement. In another embodiment, this is a therapeutic targets, and includes, a gene and its associated mRNA, mRNA isoforms thereof, protein, protein isoforms thereof, or post-translational modifications of said protein.

The term pharmagenic enrichment score or PES as used herein refers to a polygenic score calculated for a pharmacologically relevant set of genes. Specifically annotating total polygenic risk for a disorder in this fashion facilitates a more therapeutically relevant implementation of this information for any given individual. The term “polygenic risk score” is used to define an individuals' risk of developing a complex disorder or progressing to a more advanced stage of a disorder, based on a large number, typically thousands, of common genetic variants each of which might have modest individual effect sizes contribute to the disease or its progression, but in aggregate have significant predicting value. In the present case, polygenic risk score may be used to predict the likelihood that an individual will develop a complex disorder using common single nucleotide SNPs associated with the complex disorder. However, genome-wide polygenic risk score (as a biologically unannotated instrument) does not necessarily provide insight into pathways suitable for pharmacologically intervention in individuals.

In accordance with the methods disclosed herein, an elevated PES for a given pharmacologically relevant pathway is indicative that the subject will be sensitive to a therapeutic agent that is known to interact with the pharmaceutically relevant pathway. As described elsewhere herein, elevated PES is not significantly related to polygenic risk. Accordingly, the PES approach can capture latent enrichment of polygenic signal in pathways relevant to pharmaceutical actions in subjects with a low overall trait PRS relative to others with the same complex disorder phenotype.

In an embodiment, PES is calculated from SNPs mapped to genes which form the candidate pharmacologically actionable geneset. This may comprise model (1) which sums the statistical effect size of each variant in the geneset multiplied by the allele count (dosage) for said variant. For example, for individual i, let [symbol] denote the statistical effect size from the GWAS for each variant j in the geneset, multiplied by the dosage (G) of j in i.

$$P E {S}_{i} = Σ ( {\hat{β}}_{J} \times {G}_{i j} ) \qquad ( 1 )$$

The term “reference predictive polygenic score” is interchangeable with the terms “reference pharmagenic enrichment score” or “reference PES”. In an illustrative example, the comparison may be carried out using a reference predictive polygenic score that is representative of a known or predetermined predictive polygenic risk score from an individual, from a large reference cohort or a cohort of case and controls for the complex disorder phenotype in question, that is associated with sensitivity to a therapeutic agent, as described elsewhere herein.

The reference predictive polygenic score is typically a predetermined predictive polygenic score in a particular cohort or population of subjects (e.g., normal healthy controls, subjects with the complex disorder phenotype in question, subjects who had no sign of the complex disorder at the time the reference sample was obtained but who have gone on to develop the complex disorder, etc.). The reference value may be represented as an absolute number, or as a mean value (e.g., mean+/−standard deviation), such as when the reference value is derived from (i.e., representative of) a population of individuals.

Whilst persons skilled in the art would understand that using a reference predictive polygenic score that is derived from a sample population of individuals is likely to provide a more accurate representation of the predictive polygenic score in that particular population (e.g., for the purposes of the methods disclosed herein), in some embodiments, the reference predictive polygenic score can be a predictive polygenic score derived from the genome-wide variant information obtained from a single biological sample.

The pharmagenic enrichment score or PES, as described elsewhere herein, is calculated specifically relative to genes which are biologically interact with a directional anchor. The term “biologically interact” would be understood by those skilled in the art to encompass themes which include, but are not limited to, physical protein interaction, co-expression, co-occurrence in a database, and correlated expression. The two central embodiments of a directional anchor have been outlined elsewhere herein, with further elaboration in the proceeding text.

In an embodiment, a directional anchor is a therapeutic target, whereby the direction of beneficial therapeutic modulation can be predicted genetically. A therapeutic target encompasses a gene and its associated mRNA, mRNA isoforms thereof, protein, protein isoforms thereof, or post-translational modifications of said protein. This biological entity also satisfies the following criteria, i) statistically associated with the disorder or trait to be treated, ii) the direction in which modulating the therapeutic target would be therapeutically beneficial can be proposed, and iii) this entity can be modulated in said direction by some agent or other intervention. Persons skilled in the art would understand that “therapeutically beneficial” encompasses a reduction in a pathological process relative to the health of the individual to be treated. Moreover, “statistically associated” would be understood by persons skilled in the art as being related to the trait in a fashion that is greater than by chance alone, as indexed by metrics including a frequentist P value or a probabilistic Bayes' factor.

In another embodiment, a directional anchor is a therapeutic agent that would be used for the treatment of an individual. A therapeutic agent would be understood by persons skilled in the art and includes, but is not limited to, a pharmacological agent, lifestyle intervention, or non-prescription supplement.

The pharmacologically actionable gene-set around which the pharmagenic enrichment score is constructed, as described elsewhere herein, is composed of genes biologically related to the directional anchor in same fashion. Embodiments that derive these gene-sets related to the directional anchor would include: proteins predicted from experimental or in silico data to interact with the therapeutic target or a protein target of a therapeutic agent; proteins or genes that are annotated in a biological database or peer-reviewed literature article as being related to the directional anchor; genes which are statistically more likely than chance alone to be co-expressed with the directional anchor; and, genes or proteins correlated with the treatment of a directional anchor embodied as a therapeutic agent, with this treatment either in vitro, in vivo, or predicted in silico.

In an embodiment, PES is calculated from SNPs mapped to genes which form the candidate pharmacologically actionable geneset related to the directional anchor. This may comprise model (1), as defined elsewhere herein, which sums the statistical effect size of each variant in the geneset multiplied by the allele count (dosage) for said variant. In accordance with the methods disclosed herein, an elevated PES for a given pharmacologically relevant pathway is indicative that the subject will be sensitive to a therapeutic agent that comprises a directional anchor embodied as a therapeutic agent or a therapeutic agent that with a directional anchor embodied as a therapeutic target. As described elsewhere herein, elevated PES related to a directional anchor gene is not significantly related to polygenic risk.

The present disclosure will now be further described in greater detail by reference to the following specific examples, which should not be construed as in any way limiting the scope of the disclosure. In particular, this approach is amendable to the majority of disorders and traits, as outlined elsewhere herein, and thus, the named disorders and traits in the examples are only a small selection.

### EXAMPLES

### Example 1

### Identifying Directional Anchor Gene Pharmagenic Enrichment Scores for the Precision Treatment of Schizophrenia and Bipolar Disorder

Methods Relating to this Example are Henceforth Outlined:

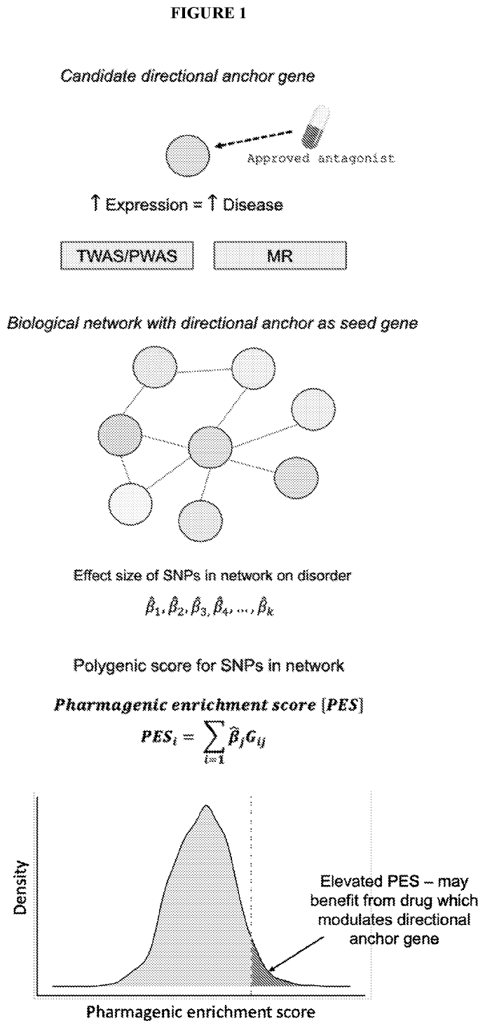

In this embodiment, the directional anchor was a therapeutic target, with a focus on therapeutic targets that are modulated by approved agents. In an example, if upregulation of a hypothetical gene, gene X, was associated with greater odds of a disease phenotype, then an antagonist of gene Xmay be clinically useful. If this gene Xantagonist is already approved for another indication, this may inform drug repurposing. However, there is immense heterogeneity between individuals for any given complex trait or disease in its genetic architecture, which often translates to highly variable clinical manifestation. We therefore state that individuals with a greater burden of disorder-associated genetic risk in the directional anchor gene, and its network of genes that physically and biologically interact with it, may benefit more specifically from a drug repurposing candidate targeting the DA-gene (pharmagenic enrichment score, PES). PES constructed from biological networks encompassing the directional anchor genes is likely to incorporate disorder-associated impacts on upstream processes that would modify the effect of a compound targeting the candidate gene, as well as downstream processes triggered by modulating the directional anchor (FIG. 1).

We obtained GWAS summary statistics for schizophrenia (SZ) and bipolar disorder (BIP) from the psychiatric genomics consortium (Schizophrenia Working Group of the Psychiatric Genomics Consortium et al., 2020, medRxiv, https://doi.org/10.1101/2020.09.12.20192922; Stahl et al., 2019, Nature Genetics, 51:793-803). The SZ GWAS was a mega-analysis of majority European ancestry cohorts and comprised 67,390 cases and 94,015 controls, whilst the European ancestry BIP GWAS mega-analysis had 20,352 cases and 31,358 controls. In addition, we also utilised the same SZ GWAS with a constituent cohort removed (Australian Schizophrenia Research Bank) when we profiled PES within that dataset, as described in a proceeding section of the methods.

A transcriptome-wide association study (TWAS) and a proteome-wide association study (PWAS) was performed of SZ and BIP by leveraging genetically imputed models of mRNA and protein expression, respectively. Specifically, we utilised the FUSION approach for TWAS/PWAS, which would be understood by those skilled in the art (Gusev et al., 2016, Nature Genetics, 48:245-252). Expression weights for the TWAS were derived from postmortem brain (GTEx v7, PsychENCODE) and whole blood (GTEx v7), whilst protein expression weights were similarly from postmortem brain (ROSMAP) and whole blood (ARIC). The FUSION methodology integrates SNP-effects from the model of genetically predicted expression with the effects of the same SNPs on SZ or BIP, after accounting for linkage disequilibrium, such that the TWAS Z score can be conceptualized measure of genetic covariance between mRNA or protein expression of the gene and the GWAS trait of interest. We utilised a conservative method for multiple-testing correction whereby the Bonferroni methodology was implemented to divide the alpha level (0.05) by the total number of significantly cis-heritable models of genetically regulated expression (GReX) tested from any brain tissue considered or whole blood. Several genes had GReX available in multiple-tissues, thus rendering Bonferroni correction conservative, however, we implemented this approach to capture only the most confidently associated genes that could constitute drug-repurposing candidates. For candidate directional anchor genes derived from TWAS/PWAS, we probabilistically finemapped those regions using the FOCUS methodology using the default prior (p=1×10⁻³) and prior variance (nσ²=40) to approximate Bayes' factors such that the posterior inclusion probability (PIP) of each gene being member of a credible set with 90% probability of containing the causal gene could be derived (Mancuso et al., 2019, Nature Genetics, 51:675-682). We also investigated the impact of using a more conservative prior as outlined in the supplementary text. Moreover, we tested whether SNPs that constitute the GReX model and either SZ or BIP displayed statistical colocalisation with the coloc package as implemented by FUSION.

In addition, we leveraged variants strongly correlated with mRNA (expression quantitative trait loci—eQTL) and protein expression (protein expression quantitative trait loci—pQTL), respectively, as instrumental variables (IVs) in a two-sample Mendelian randomisation (MR) analysis (Hermani et al, 2018, eLife, 7:e34408). Analogous to the TWAS/PWAS, eQTL/pQTL were derived from brain (MetaBrain, ROSMAP) and blood (eQTLGen, Zhang et al., 2020, Nature Genetics, 52:1122-1131). Strict selection criteria were implemented to select suitable IVs, including only retaining independent genome-wide significant (P<5×10⁻⁸) SNPs that were associated with three or less mRNA/proteins in each relevant tissue/study. Moreover, we utilised a more stringent LD clumping procedure for eQTLs given the greater power and sample sizes for these datasets also results in immense pleiotropy amongst the SNP effects on mRNA by only selecting the most significant independent SNPs using one megabase clumps, with LD estimated using the 1000 genomes phase 3 panel. The effect of mRNA or protein expression for any given gene on SZ or BIP was estimated using the Wald ratio (single IV) or an inverse-variance weighted estimator (multiple IVs, with fixed effects due to the small number of IVs). As in the TWAS/PWAS, we utilised Bonferroni correction across all tissues in the mRNA and protein analyses respectively and then sought to identify candidate directional anchor genes from these signals. For any candidate directional anchor genes, that is, where an approved drug was predicted to reverse the odds increasing direction of expression, we performed a series of sensitivity analyses. Briefly, these involved: assessing the genomic locus of the IV SNP and which other genes, if any, it was associated with, testing evidence for a shared causal variant via colocalisation using default priors and conducting a phenome-wide Mendelian randomisation analysis (MR-pheWAS) using SNP effects from each trait in the IEUGWAS database. The above MR and sensitivity analyses were performed using the R packages TwoSampleMR v0.5.5, ieugwasr v0.1.5, and coloc v4.0.4.

We searched genes prioritised from the TWAS/PWAS or MR analyses in the Drug-gene interaction database (DGIdb v4.2.0—accessed April 2021) to identify approved compounds that could reverse the odds increasing direction of expression for SZ or BIP. DGIdb combines data from databases such as DrugBank, as well as curated literature sources. We defined high confidence drug-gene interactions as those reported in DrugBank as well as at least one other database or literature source.

Protein-protein interaction data was downloaded from the STRING database vI1. We utilised each of the six candidate directional anchor genes as a seed gene, separately, and constructed a network of genes predicted to interact with the seed gene by retaining high confidence edges (confidence score>0.7) derived from experimental evidence or curated protein-complex and pathway databases, as this is generally considered the most rigorous evidence from STRING. We then tested which gene-sets curated by the g:Profiler (version e104_eg51_p15_3922dba) resource (GO, KEGG, Reactome, WikiPathways, TRANSFAC, miRTarBase, Human Protein Atlas, CORUM, and Human phenotype ontology) were overrepresented amongst the genes in each network, using the g:SCS (set counts and sizes) multiple-testing correction method implemented by g:Profiler that has been shown to better account for the complex, overlapping nature of these data. We considered a corrected P value<0.05 as significant. We then tested the association of the genes in each of these networks, with and without the gene removed, with the common variant signal in the SZ and BIP GWAS using MAGMA v1.09. Briefly, SNP-wise P values were aggregated at gene-level, with SNPs annotated to genes using two different sets of genic boundary extensions to capture potential regulatory variation, conservative (5 kilobases (kb) upstream, 1.5 kb downstream), and liberal (35 kb upstream, 10 kb downstream). Gene-set association is implemented by MAGMA using linear regression, whereby the probit transformed genic P values (Z scores) are the outcome with a binary explanatory variable indicating whether a gene is in the set to be tested (β_{S}), covaried for other confounders like gene size, as described previously. The test statistic of interest is a one-sided test of whether β_{S}>0, and thus, quantifies if the genes in the set are more associated than all other genes. We also investigated the association of the approximately 34,000 gene-sets collated by g:Profiler, such that we could demonstrate whether gene-sets overrepresented in each network were also associated with SZ or BIP.

We sought to utilise variants annotated to the genes within the network of each candidate directional anchor genes as to develop pharmagenic enrichment scores for SZ and BIP, respectively. As described previously, a PES is analogous to a genome-wide PRS in its derivation, with the key difference that it only utilises variants mapped to the gene-set of interest (equation 1) [18]. Specifically, a PES profile in individual i comprises sum of the effect size of j variants from the GWAS (\hat{β}ⱼ) annotated to at least one gene in set M, multiplied by the allelic dosage under an additive model (GᵢⱼϵG=0, 1, 2). PESᵢ=Σⱼ₌₁^{M}\hat{β}ⱼGᵢⱼ [1]. The genome wide PRS for SZ and BIP are essentially the same model but M incorporates the entire genome. In accordance with the MAGMA analyses, we tested two genic boundary configurations for evaluating the best performing PES for each directional anchor gene network—conservative and liberal. Our previous PES related approaches utilised the LD clumping and thresholding (C+T) approach, whereby SNPs are ‘clumped’ such that the retained SNPs are largely independent and ‘thresholded’ based on their association P-value in the GWAS. In each case the threshold is set at the level the optimal for the druggable gene-set association at the population level. However, given we selected the gene-sets in this study based on interactions with the candidate directional anchor gene we tested four different P value thresholds (P_{T}ϵT={0.005, 0.05, 0.5, 1}), which represent a model with all SNPs, nominally significant SNPs, and a threshold an order of magnitude above or below the nominal threshold. We utilised PRSice-2 v2.3.3 (linux) for the C+T models. In addition, we also utilised a penalised regression framework to shrink SNP effect sizes to optimize the model for each PES, as implemented by the standalone version of lassosum v0.4.5. The implementation for this method has been outlined extensively elsewhere, with the optimal tuning parameter (λ) based on the score that displays the highest correlation with the phenotype and the best performing constraint parameter (s) chosen from a range of apriori specified values to decrease computational burden (0.2, 0.5, 0.9, and 1).

We utilised the prospective UK Biobank (UKBB) cohort to define the best performing PES for each directional anchor gene network. Our group has previously processed the UKBB genotype data such that unrelated individuals of white British ancestry were retained, along with other sample and variant level quality control considerations applied [20]. As a result, the composition of the full UKBB cohort in this study was 336,896participants for which up to 13,568,914 autosomal variants were available (imputation INFO>0.8). SZ and BIP cases were defined in the UKBB using a combination of self-report data both from the general assessment visit and the mental health questionnaire (MHQ), along with hospital inpatient data (primary or secondary ICD-10 codes). In total, there we 631 UKBB participants from the study cohort defined as having SZ, with 1657 BIP cases identified. The controls were double the number of the respective case cohorts randomly, and independently for SZ and BIP, derived from 75,201 individuals with genotype data that completed the MHQ and did not self-report any mental illness. The full complement of SZ cases with the aforementioned controls (N=1262) was utilised as the training set for the SZ scores given the relatively small number of cases. As a result, we utilised the Australian Schizophrenia Research Bank (ASRB) cohort as a validation set to attempt to replicate the associations observed with the scores (Loughland et al., 2010, Aust N Z JPsychiatry, 44 :1029-1035). The ASRB was a component of the PGC3 SZ GWAS, and thus, we retrained all the best performing PES scores using summary statistics with the ASRB cohort removed before testing them in that dataset. The BIP analyses employed a 70/30 split for the training and validation cohort in the UKBB, with double the number of independent MHQ derived healthy controls utilised for each case-set. Further information regarding the demographic composition of these cohorts is provided in the supplementary text. The PES and PRS constructing using the C+T configurations and penalised regression were scaled to have a mean of zero and unit variance before evaluating their association with SZ or BIP for the respective scores in the UKBB training cohorts using binomial logistic regression covaried for sex, age, genotyping batch, and five SNP derived principal components. The optimal PES for each network was selected for each disorder separately by calculating the variance explained on the liability scale (Nagelkerke's R²), assuming a 0.7% and 1% prevalence for SZ and BIP, respectively. These PES/PRS that explained the most phenotypic variance were then profiled and tested in the validation sets. For PES that were significantly associated with either disorder, we conservatively constructed another model that also included genome wide PRS, with a χ² test of residual deviance performed to ascertain whether adding the PES in addition to the PRS significantly improved model fit. Correlations (Pearson's) amongst scaled the PES and PRS were visualised using the corrplot package v0.84. Individuals with at least one elevated PES in the training cohorts (highest decile) were identified, with this binary variable tested for association with SZ or BIP using another logistic regression model. Finally, we also considered residualised PES, whereby the residuals were extracted and scaled from a linear model that regressed genome wide PRS against principal components and genotyping batch on the score in question. All analyses described in this section were performed utilising the programming language R (version 3.6.0).

We then wished to investigate the correlations between the best performing PES for each network and i) blood or urine biochemical traits, and ii) self-reported mental health disorders besides SZ or BIP. The biochemical analyses were performed in up to 70,625 individuals who did not self-report any mental health disorders in the MHQ and were also not included in the SZ or BIP training/validation sets as controls. There were 33 biochemical traits tested (raw measured values) in a linear model with each PES or PRS as an explanatory variable covaried for sex, age, sex x age, age², 10 principal components, and genotyping batch. We also performed sex-stratified analyses, with oestradiol additionally considered in females. A number of sensitivity analyses were performed for PES-biochemical trait pairs that were significantly correlated after FDR correction—i) adjustment for genome-wide PRS, ii) natural log transformation of the biochemical outcome variable, iii) inverse-rank normal transformed residuals as the outcome variable from a model that regressed sex, sex x age, and age², and iv) adjustment for statin use (given the number of lipid related signals uncovered). These correlations are observational in nature, and thus, there are several other potential confounders that could be considered—however, given the potential biases induced by adjusting for heritable covariates, we utilised the above strategies as a baseline suite of sensitivity analyses. A specific test of sexual dimorphisms between the regression results in males and females was also performed based on the sex-specific regression estimates and standard errors (Martin et al., 2021, Biological Psychiatry, 89:1127-1137). Moreover, we then evaluated the association between each score and 14 non-SZ or BIP mental health disorders which individuals who completed the MHQ were asked to self-report. In all instances, we used the 70,625 individuals who did not self-report any mental disorders as the controls in binomial logistic regression models covaried for the same terms as in the biochemical analyses.

The findings and data related to this example are detailed henceforth.

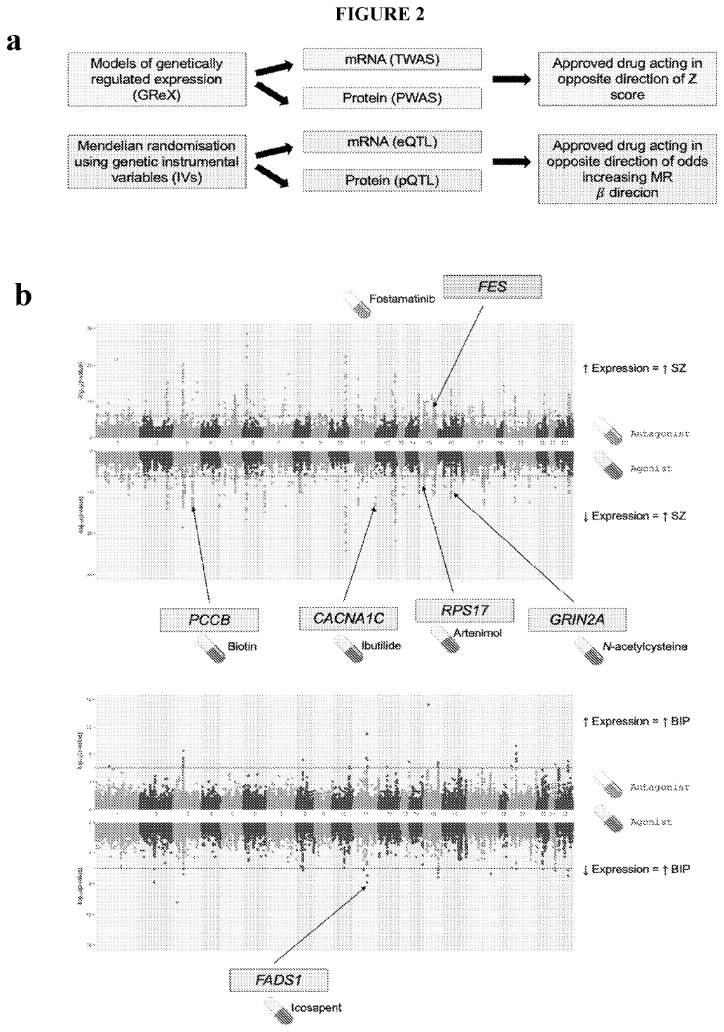

We sought to identify candidate directional anchor genes for SZ or BIP by integrating GWAS summary statistics for these traits with transcriptomic and proteomic data collected from either blood or post-mortem brain (FIG. 2a). Specifically, we utilised genetically imputed models of mRNA or protein expression to conduct a TWAS and PWAS, respectively. Genome-wide significant eQTLs and pQTLs were also leveraged as instrumental variables in a more conservative two-sample Mendelian randomisation analysis to explicitly test for any causal effects of mRNA or protein expression. After implementing Bonferroni correction within each analysis set (TWAS, PWAS, eQTL-MR, pQTL-MR), we found several genes for which their expression was associated with at least one of the psychiatric phenotypes at the mRNA or protein level that was also putatively modulated by an approved compound in a risk decreasing direction. There were 13 druggable genes from TWAS for which the direction of genetically predicted mRNA expression correlated with SZ could be pharmacologically counteracted, whilst there were two such genes for BIP, some examples of which visualised in FIG. 2b. For instance, imputed mRNA expression of the calcium voltage-gated channel subunit gene CACNA1C was negatively correlated with SZ (P=3.65×10⁻¹⁵), and thus, an activator of this gene like the antiarrhythmic agent Ibutilide may be a repurposing candidate for SZ, with this gene also trending towards association with BIP in the same direction (P=3.18×10⁻⁵). We compared the TWAS results to that of a PWAS using data from blood or brain tissue, although the number of proteins assayed in these studies was considerably smaller than that of the number of mRNA available, and thus, most of the candidate genes derived using TWAS did not have protein measurements available for a direct comparison of the effect of protein expression relative to mRNA. However, there were two Bonferroni significant TWAS genes that represented a plausible repurposing candidate with protein expression data available (NEK4 and CTSS), with both of these genes showing a similar strength of association in the PWAS. Any given gene displaying an association between genetically predicted mRNA or protein expression and the trait of interest does not necessarily imply it is the causal gene at that locus due to factors like LD complexity and other phenomena like co-regulation, as described extensively elsewhere. This is critical for using these approaches for drug repurposing as we wish to target the genes that actually are responsible for the genetic association with expression in that region. As a result, we implemented a Bayesian finemapping procedure for each TWAS candidate gene to identify plausible causal genes in each locus. We found four repurposing candidate genes for SZ with strong evidence for membership of a credible set with 90% probability of containing the causal gene (PIP>0.8—PCCB, GRIN2A, FES, and CACNAID). However, CACNAID was excluded from further analyses due to the poor performance of its imputed model and complexity of its locus on chromosome three, as outlined more extensively in the supplementary text. We then considered a more lenient posterior inclusion probability of 0.4, which identified two more genes for SZ (CACNA1C and RPS17), and a BIP gene (FADS1). Colocalisation analyses were also performed to test a related, but distinct hypothesis that the GWAS signal and SNP weights in the expression model share an underlying single causal variant. Interestingly, for the genes selected using the lower confidence PIP>0.4 threshold, we found strong evidence for a shared causal variant (PP_{H4}>0.9), supporting their inclusion as putative drug repurposing targets. We did not consider the two genes shared with the PWAS any further as they did not display strong finemapping support in the TWAS, which is a more accurate representation of any given locus due to the more expansive number of genes with RNAseq available. In summary, using a genetically imputed expression approach we identified five candidate directional anchor genes for SZ and one for BIP (Table 1). For example, imputed GRIN2A mRNA expression was negatively correlated with SZ (P=1.44×10⁻⁹), with a trend also observed for BIP (P=5.07×10⁻³), with compounds of interest in psychiatry, such as N-acetylcysteine, known to agonise this subunit (REF).

TABLE 1
Candidate directional anchor genes (therapeutic targets) for schizophrenia
and bipolar along with their associated drug repurposing candidates.

| | | | Repurposing | |
|---|---|---|---|---|
| Gene | Disorder | Protective direction | candidates | Sensitivity analyses |
| PCCB | SZ | Increased expression | Biotin | High confidence causal |
| | | | | gene from TWAS (PIP > |
| | | | | 0.8). Further supported by |
| | | | | MR |
| FADS1 | BIP | Increased expression | Icosapent | Moderate confidence |
| | | | Linolenic acid | causal gene from TWAS |
| | | | | (PIP > 0.4, additionally |
| | | | | supported by colocalisation - |
| | | | | PP_{H4} > 0.8). Further |
| | | | | supported by MR |
| GRIN2A | SZ | Increased expression | N-acetylcysteine | High confidence causal |
| | | | Glycine | gene from TWAS (PIP > |
| | | | | 0.8). |
| CACNA1C | SZ | Increased expression | Ibutilide | Moderate confidence |
| | | | | causal gene from TWAS |
| | | | | (PIP > 0.4, additionally |
| | | | | supported by colocalisation - |
| | | | | PP_{H4} > 0.8). |
| RPS17 | SZ | Increased expression | Artenimol | Moderate confidence |
| | | | | causal gene from TWAS |
| | | | | (PIP > 0.4, additionally |
| | | | | supported by colocalisation - |
| | | | | PP_{H4} > 0.8). |
| FES | SZ | Decreased expression | Fostamatinib | High confidence causal |
| | | | Lorlatinib | gene from TWAS (PIP > |
| | | | | 0.8). |

Moreover, we then utilised eQTL and pQTL as IVs in a Mendelian randomisation analysis to priortise candidate directional anchor genes, which seeks to estimate the causal effect of mRNA or protein expression on either disorder outcome, given more onerous assumptions are met (Supplementary Materials, Supplementary Tables 7-10). This is critical as the use of molecular QTLs related to variables like mRNA expression as IVs is challenging due to LD complexity and the potential effect of QTLs on multiple genes [28, 46]. As a result, we sought to complement the above discovery orientated TWAS/PWAS with more conservative selection criteria for an eQTL or pQTL to be an IV, particularly in the case of eQTLs where sample sizes for some tissues are now very large. Independent SNPs (LD r²<0.001) acting as eQTLs or pQTLs at a threshold of genome-wide significance (P<5×10⁻⁸) were selected from post-mortem brain or blood, as outlined in the methods and supplementary materials. Due to the conservative nature of these analyses, many of the genes considered in the TWAS/PWAS did not have a suitable IV available, whilst conversely, a small number of genes that did not display adequate multivariate cis-heritability in the TWAS/PWAS weights could now be included. The mRNA models after Bonferroni correction uncovered four genes for which expression exerted a potential causal effect on SZ or BIP with a suitable compound approved to reverse the odds-increasing direction of effect. There were three for SZ (PCCB, NEK1, and PTK2B), as well as FADS1 for bipolar. Interestingly, PCCB and FADS1 overlapped with the TWAS results—as an example, each standard deviation increase in cortical FADS1 expression was associated with an approximately 15.23% [95% CI. 8.69%, 21.77%] decrease in the odds of BIP, which could be accentuated by an FADS1 agonist like the omega-3 fatty acid supplement icosapent (Ethyl eicosapentaenoic acid). We then performed a series of sensitivity analyses to assess IV validity and for evidence of confounding pleiotropy. These analyses supported PCCB and FADS1 as candidate directional anchor genes. The index IV-SNPs mapped to PCCB and FADS1 expression, respectively, was then utilised to perform a phenome-wide scan spanning over 10,000 GWAS of the effect of expression of these two genes using SNP effect sizes the IEUGWAS database. Firstly, we found that increased cortical expression of PCCB, which was associated with deceased odds of SZ from a previous GWAS, was also linked to a reduction in other psychiatric phenotypes from self-reported UK Biobank GWAS such as worry, neuroticism, nervousness, and tenseness, supporting the utility of a PCCB agonist, like biotin, as a repurposing candidate. Secondly, the phenome-wide data for increased cortical FADS1 expression demonstrated, as expected, a strong effect on lipids, including increased HDL and decreased triglycerides. We did find one potential other candidate for BIP using the pQTL approach (MAP2K2), however, given this was a trans-pQTL, we did not consider it further as we wished to retain only the most biologically confident associations. As a result, the MR approach did not add any additional candidate directional anchor genes but provided more support to PCCB and FADS1. A less conservative MR paradigm in terms of IV selection would likely yield more genes but as our TWAS/PWAS analyses were already discovery focused, we believe this is appropriate given the underlying assumptions of MR. We summarize the candidate directional anchor genes in table 1.

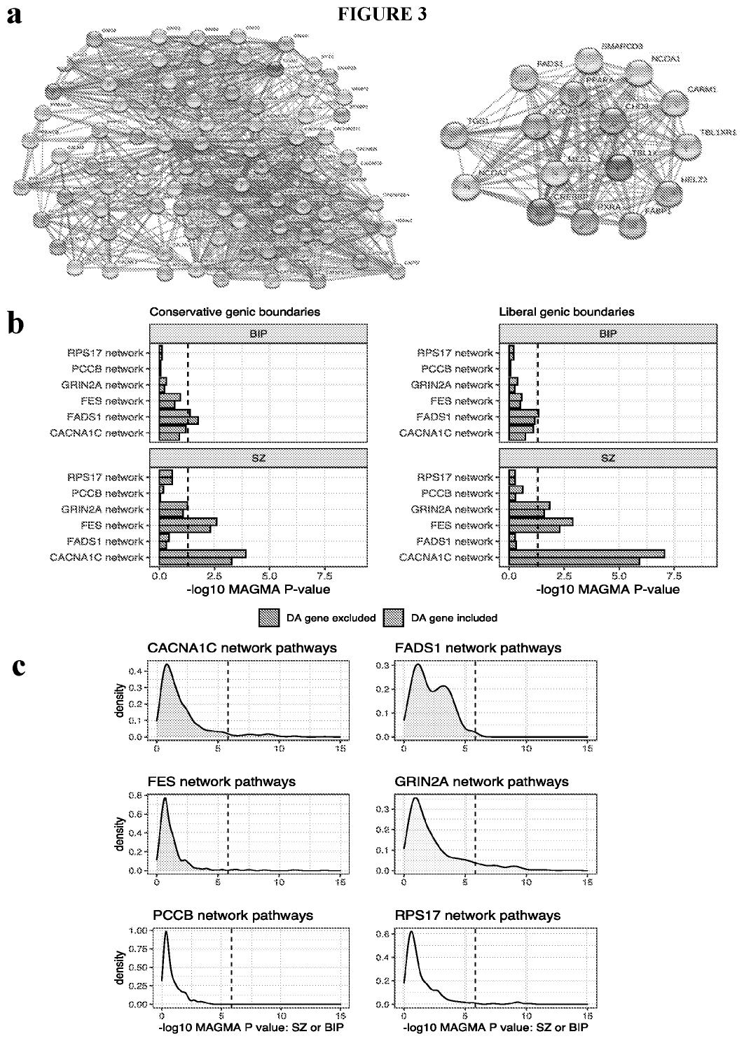

We sought to define a network of genes that display high confidence interactions with each candidate directional anchor gene using data from the STRING database, such that we can then construct a pharmagenic enrichment score using variants annotated to these genes. The number of direct interactions identified for each of the six candidate genes, excluding the gene itself, were as follows: CACNA1C network (83 genes), FADS1 network (16 genes), FES network (37 genes), GRIN2A network (54 genes), PCCB network (26 genes), and RPS17 network (254 genes). All of these networks displayed significantly more interactions than what would be expected by chance alone (P<1×10⁻¹⁶), with an example of two of these networks (CACNA1C and FADS1) visualised in FIG. 3a. These networks likely represent heterogenous biological processes in which the directional anchor gene may participate, and thus, we sought to better understand the biology of these interacting genes by testing their overrepresentation within biological pathways and other ontological gene-sets. The six directional anchor gene networks each displayed overrepresentation in pathways related to the known biology of the candidate gene. For instance, the CACNA1C network genes were enriched within several hundred gene-sets, many of which related to neuronal calcium channel biology along with systemic processes known to involve calcium signalling such as pancreatic insulin secretion. Furthermore, the FADS1 network genes displayed an overrepresentation in several lipid and other metabolic related pathways, whilst GRIN2A network genes demonstrated a strong link to neuronal biology.

We then tested whether there was enrichment of the common variant signal for SZ or BIP in any of these networks, with and without the directional anchor gene included, using MAGMA (FIG. 3b). The CACNA1C network was strongly associated with SZ (P=8.87×10⁻⁸), even after removing CACNA1C itself (P=1.19×10⁻⁶). The FES and GRIN2A networks demonstrated a nominal enrichment of the SZ common variant signal relative to all other genes, P=1.28×10⁻³ and P=0.014, respectively, remaining significant upon removing the relevant directional anchor genes. None of the other networks were associated with SZ when considering all genes, with only the FADS1 network demonstrating a nominal association with BIP (P=0.04). Given that these networks represent several different biological processes, we further hypothesized that specific gene-sets for which they were overrepresented may specifically display a stronger association with SZ or BIP. Indeed, we show that all of the networks had at least one overrepresented pathway that was associated with SZ or BIP using Bonferroni (FWER<0.05) and Benjamini-Hochberg (FDR<0.05) correction, with the exception of the sets enriched in the PCCB network that only survived correction using FDR. Kernel density estimation plots of the MAGMA gene-set association P values are visualised in FIG. 3c, which show pathway-associations reaching these thresholds. We briefly describe the results for the CACNA1C and GRIN2A networks below for illustration. Pathways overrepresented in the CACNA1C network related to calcium channel activity displayed strong association with SZ, for instance, voltage gated calcium channel process (P=2.80×10⁻¹, q=6.83×10⁻⁷), whilst the regulation insulin secretion pathway that also was enriched in the network was associated with SZ and trended towards surviving multiple testing correction for BIP. GRIN2A network members also displayed an enrichment amongst several neuronal pathways strongly associated with SZ, such as synaptic signalling (P=3.88×10⁻¹⁰, q=2.82×10⁻⁵). Taken together, these results suggest that pathways in which genes in each network participate are associated with psychiatric illness and reinforces the biological salience of these networks.

Pharmagenic enrichment scores (PES) were then constructed for the genes in each directional anchor gene network using SNP weights for SZ and BIP, respectively. SZ and BIP PES were considered for all six networks given the high genetic correlation between SZ and BIP, as well as extensive phenotypic overlap. We defined a training set of SZ (N=631) and BIP cases (N=1161) in the UK Biobank, with double the number of controls randomly, and independently, selected from individuals with no self-reported mental health conditions for each training set. Two methods were utilised to find the most parsimonious PES profile for each network, along with a genome-wide PRS for SZ and BIP—clumping and thresholding (C+T), and penalised regression (Table 2).

TABLE 2
Characteristics of the best performing schizophrenia and bipolar genome wide PRS along
with a pharmagenic enrichment score for each directional anchor gene network

| | | | | | |
|---|---|---|---|---|---|
| Score | N_{SNPs}¹ | Beta (SE)² | P | R² | Type³ |

Bipolar disorder

| | | | | | |
|---|---|---|---|---|---|
| PRS | 1096096 | 0.62 (0.04) | 2.91 × 10⁻⁵¹ | 4.96% | Penalised regression |
| CACNA1C network | 396 | 0.11 (0.04) | 2.97 × 10⁻³ | 0.16% | Penalised regression |
| FADS1 network | 196 | 0.06 (0.04) | 0.1 | 0.05% | C + T |
| FES network | 5 | 0.10 (0.04) | 4.87 × 10⁻³ | 0.14% | Penalised regression |
| GRIN2A network | 143 | 0.17 (0.04) | 3.81 × 10⁻⁶ | 0.39% | Penalised regression |
| PCCB network | 520 | 0.14 (0.04) | 2.22 × 10⁻⁴ | 0.25% | Penalised regression |
| RPS17 network | 13724 | 0.15 (0.04) | 1.07 × 10⁻⁴ | 0.28% | Penalised regression |

Schizophrenia

| | | | | | |
|---|---|---|---|---|---|
| PRS | 1861450 | 1.01 (0.07) | 2.65 × 10⁻⁵¹ | 9.55% | Penalised regression |
| CACNA1C network | 128 | 0.09 (0.05) | 0.1 | 0.09% | C + T |
| FADS1 network | 36 | 0.04 (0.05) | 0.4 | 0.02% | C + T |
| FES network | 141 | 0.17 (0.05) | 1.41 × 10⁻³ | 0.32% | C + T |
| GRIN2A network | 5037 | 0.18 (0.05) | 9.23 × 10⁻⁴ | 0.35% | Penalised regression |
| PCCB network | 2393 | 0.07 (0.05) | 0.16 | 0.06% | Penalised regression |
| RPS17 network | 77 | 0.15 (0.05) | 3.53 × 10⁻³ | 0.27% | Penalised regression |

¹SNPs with a non-zero coefficient after the reweighting in the penalised regression model or independent SNPs after linkage disequilibrium clumping and thresholding (C + T).
²Bipolar disorder or schizophrenia log odds per standard deviation increase in the score (standard error)
³The two models evaluated were clumping and thresholding (C + T) or penalised regression (as implemented by the lassosum package)

In the SZ cohort, there were three network SZ PES which were significantly associated with increased odds of SZ after multiple testing correction including networks for FES, GRIN2A, and RPS17 (Table 2). In the GRIN2A network PES featuring 5037 variants constructed using penalised regression explained approximately 0.35% of phenotypic variance on the liability scale (OR per SD in score=1.19 [95% CI. 1.09, 1.29], P=9.23×10⁻⁴). We then conservatively adjusted for the best performing genome-wide SZ PRS and found that the GRIN2A network PES remained significantly associated with SZ. In the FES and RPS17 networks, their PES were just below the threshold for significance after PRS adjustment. It is notable that the SZ network PES profiles were only marginally correlated with genome-wide SZ PRS (all r<0.11), which suggests these scores may capture biologically aggregated risk which is distinct from the undifferentiated genome-wide signal. Individuals with elevated SZ directional anchor network PES were then defined as those with scores in the highest decile (≥90^{th} percentile). The majority of SZ cases (53.72%) had at least one elevated PES, with a significant enrichment of SZ cases amongst individuals with an elevated PES, even after covariation for genome-wide PRS−OR=1.45 [95% CI: 1.22, 1.67], P=1.57×10⁻³. Interestingly, amongst individuals in this cohort with relatively low SZ PRS (lowest decile), 12 out of the 19 SZ cases with low PRS had an elevated PES (63%), with a nominally significant association remaining between elevated PES and SZ amongst those with low genome wide PRS (P=0.027). Upon considering only SZ cases in terms of low PRS, we found that 46.88% had at least one elevated PES. Taken together, these data suggest that some individuals with otherwise low SZ PRS may have localised genetic risk within particular biologically related networks. Given the relatively small number of SZ cases in the UKBB, we sought to replicate our results using an independent case-control cohort from the ASRB (N_{Cases}=425, N_{Controls}=251) rather than splitting the UKBB cohort into a training and validation set. The PES and PRS models were retrained in the UKBB from the same GWAS with the ASRB cohort removed. We were able to nominally replicate the association of the FES network PES with SZ in the ASRB (OR per SD=1.21 [95% CI: 1.04, 1.38], P=0.024), whilst the observed association between the GRIN2A and RPS17 network PES and SZ and in the UKBB was not replicated.

BIP PES within these networks was then profiled in the UKBB training set (Table 2). Interestingly, there were more of the directional anchor gene network PES associated with BIP than SZ, which may reflect that larger number of BIP cases in the UKBB, and thus, greater statistical power. Specifically, all of the network BIP PES were significantly higher in cases, with the exception of the FADS1 network PES for which there was only a trend towards significance. Analogous to the SZ cohort, the GRIN2A network PES explained the most phenotypic variance on the liability scale (0.39%), with each SD in the score associated with approximately an 19% [95% CI: 12%, 26%] increase in the odds of BIP. Moreover, adjustment for BIP genome wide PRS did not ablate the significance of the GRIN2A network, RPS17 network PES, and FESnetwork PES, whilst the PCCB network PES trended towards significance (P=0.1) after PRS covariation. The correlations between each PES and BIP PRS were also small, however, the RPS17 network PES (r=0.13), CACNA1C network PES (r=0.14), and PCCB network PES (r=0.13) were slightly larger in terms of their PRS correlation than what was observed for the SZ scores. We then investigated the characteristics of individuals with elevated BIP PES and found like SZ that almost half of the BIP cases (49.1%) had at least one PES greater than or equal to the 90^{th} percentile. There was also enrichment of BIP cases amongst participants with an elevated PES after adjusting for BIP PRS (OR=1.19 [95% CI: 1.04, 1.34], P=0.027). Considering BIP cases in the lowest decile of the BIP PRS distribution, 36% of them had at least one top decile PES despite their low genome-wide burden, although unlike SZ the association between elevated PES and case-status in this subcohort was not statistically significant. An independent BIP case-control cohort from the UKBB was utilised to attempt to replicate these associations, and we found that the network RPS17 PES was significantly enriched in BIP cases in this validation cohort: whilst there was a trend for the GRIN2A network PES (P=0.052).

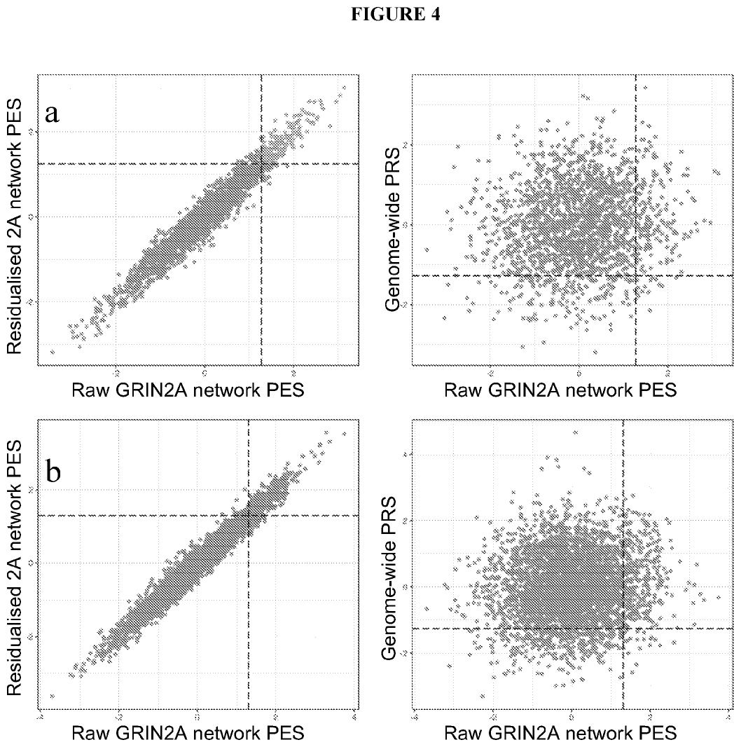

The GRIN2A network PES explained the most phenotypic variance for SZ and BIP, and survived covariation for genome-wide PRS—thus, we wanted to test whether constructing a PES for this network with the GRIN2A gene excluded would still be associated. In other words, we investigated whether there was an effect from variants mapped to the network without the directional anchor gene itself. Indeed, we did find that the GRIN2A network PES with the GRIN2A gene removed was still significantly enriched in both SZ and BIP (P_{SZ}=9.23×10⁻⁴ and P_{BIP}=3.81×10⁻⁶). The relationship between genome-wide PRS and this PES was also examined in further detail by constructing a ‘residualised PES’ whereby we obtained the normalized residuals from a model that regressed SNP derived principal components, genotyping batch, and genome-wide PRS for BIP and SZ, respectively, on the GRIN2A network PES for either disorder. We posit that the individuals with an elevated residualised PES are more likely to represent true enrichment in that network given that the effect of the genome wide PRS, along with variables related to technical artefacts and population stratification, have been adjusted for. Encouragingly, we find that the correlation between the raw GRIN2A network PES for either disorder and their respective residualised PES were highly concordant, with the majority of individuals with an elevated GRIN2A PES (≥90^{th} percentile) also in that same quantile for the residualised PES (FIG. 4a-b).

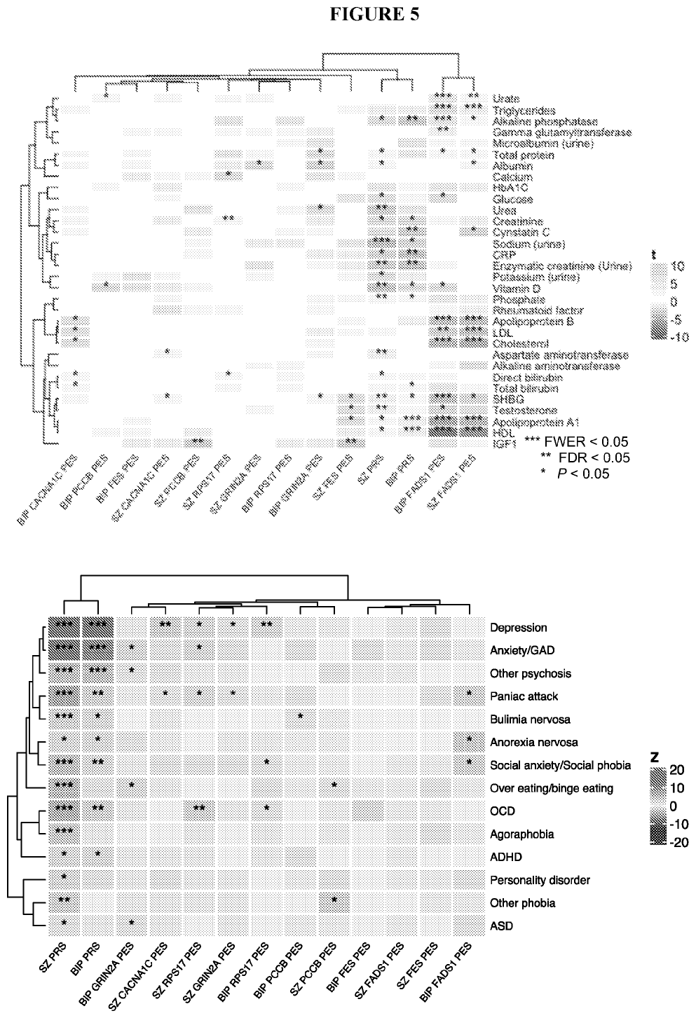

We then investigated the association of the directional anchor gene PES in an independent subset of the UKBB with other mental health phenotypes and systemic biochemical measures (FIG. 5). The correlation profile of each PES relative to these phenotypes may also support its clinical utility, whilst it also provides an opportunity further establish what distinct properties these scores have from a genome wide PRS. Firstly, all SZ and BIP network PES, along with their respective PRS, were regressed against 33 blood and urine measures in up to 70,625 individuals, whilst oestradiol in females was also additionally considered in a sex stratified analysis (FIG. 5a). In both sexes, we found that the FADS1 network PES for SZ and BIP was significantly correlated with lipid related traits after conservative Bonferroni correction for all PES/PES-trait pairs tested (P<1.11×10⁻⁴). For instance, these FADS1 network PES were negatively correlated with HDL cholesterol and apolipoprotein A1 levels, whilst an increase in that same PES was associated with higher measured triglycerides. The FADS1 network PES were also significantly associated other non-lipid biochemical traits including alkaline phosphatase, sex-hormone binding globulin (SHBG), and urate. Notably, adjusting these models for a genome-wide PRS for SZ or BIP, respectively, did not ablate the association, supporting that these signals are not just a product of genome-wide polygenic inflation, whilst there was some evidence that PRS actually was correlated in the opposite direction with lipids to that of the PES. Given the strong lipid related signals, we also adjusted for statin use in an additional sensitivity analysis, but this similarly did not markedly impact the findings. Using less stringent FDR correction (FDR<0.05), revealed more PES associated with biochemical measures, such as a negative correlation between the SZ PCCB network PES and FES network PES as insulin-like growth factor 1 (IGF1), as well positive correlation between SZ RPS17 network PES and creatinine. There was no direct effect of SZ or BIP PRS on IGF-1 or creatinine, with FES-related tyrosine kinase activity previously shown in the literature to be associated with IGF-1 biology. Sex stratified analyses identified even more PES associated with a biochemical trait—for example, in males the SZ PCCB network PES was positively correlated with SHBG, which interestingly is in the opposite direction to the correlation of SHBG observed with the FADS1 network PES, further highlighting biological heterogeneity amongst different networks. The BIP CACNA1C network PES in males was also positively correlated with direct bilirubin using an FDR cut-off, whilst the BIP GRIN2A network PES was negatively correlated with measured total protein. Finally, we formally tested for evidence of sexual dimorphic effects of PES/PRS on each biochemical measure and revealed nominal evidence of heterogeneity between sexes in these associations for some traits such as the effect of the CACNA1C network PES on direct bilirubin.

We also performed a phenome-wide association study of each score with 14 self-reported mental health disorders in the UKBB cohort, excluding SZ and BIP (FIG. 5). The number of cases ranged from 66 for attention deficit/hyperactivity disorder (ADHD) to 22,974 for depression, with the same cohort of 70,625 individuals without a self-reported mental health condition not featured in the SZ or BIP training/validation sets leveraged as controls. Unsurprisingly, we found that SZ and BIP PRS were strongly associated with increased odds of several mental health disorders after Bonferroni correction, but we also found network PES associated with some of these phenotypes using FDR<0.05 as the multiple-testing correction threshold. Specifically, there was an association between the SZ CACNA1C PES and increased odds of depression, whilst the SZ RPS17 network PES was associated with increased odds of self-reported OCD. These disorders were also associated with elevated SZ PRS, however, both PES remained significantly higher in those with the respective self-reported phenotypes even after covariation for the effect of the SZ PRS. There were also several other nominal associations (P<0.05), including one of particular interest in the case of the BIP FADS1 network PES, for which a higher score displayed some evidence of a protective effect on self-reported anorexia nervosa. Whilst this association does not survive multiple testing correction, and thus should be interpreted cautiously, it is notable as the FADS1 network PES was associated with lipid profiles in an analogous direction to what has previously shown to be genetically correlated with anorexia nervosa GWAS via LD score regression. In summary, these data coupled with the biochemical associations support the distinct nature of directionally anchored network PES from PRS and emphasise the unique insights that can be afforded by these partitioned scores.

### Example Two

### Precision Targeting of FTO Inhibitors in Breast Cancer Informed by the Directional Anchor Pharmagenic Enrichment Score Platform

Methods relating to this example are henceforth outlined. In this embodiment, the directional anchor is a therapeutic agent, in the form of compounds that inhibit the activity of the FTO gene.

We sought to refine the target genes of two recent, potent small-molecular inhibitors of FTO—specifically, CS1 (bisantrene) and CS2 (brequinar). RNA sequencing was performed to explore the mRNA expression correlates in vitro of CS1 and CS2, as described extensively in that study (Su et al., 2020, Cancer Cell, 38(1):79-96.el1). The human monocytic leukaemia cell line NOMO-1 was treated with both compounds individually (three replicates each) relative to four control replicates. In addition, the effect of FTO knockdown via short-hairpin RNA (shFTO) was also investigated relative to a control construct (shNS). We obtained raw count data generated by HTSeq for the aforementioned experiments by correspondence with the lead author, with data in the form of transcript-per-million uploaded to GEO for this study (GSE136204).

Data normalisation, filtration, and differential expression analyses were performed using the edgeR package version 3.34.0. We considered three different contrasts: i) CS1 treated cells vs control replicates, ii) CS2 treated cells vs control replicates, and iii) shFTO treated cells vs shNS treated control replicates. Raw counts were firstly normalised to library size, followed by removing lowly expressed genes with fewer than 10 raw counts in the smallest library via a counts-per-million thresholding approach. Data were inspected before and after the filtration step via coefficient of variation (BCV) and multidimensional scaling (MDS) plots. Differential expression for each gene that survived quality control was then performed using exact tests for differences in the means between two groups of negative-binomially distributed counts. We defined a differentially expressed gene as those which survived multiple-testing correction using the Benjamini-Hochberg false discovery rate approach at the 1% threshold and had an absolute log₂ fold change (FC)>0.6, which approximates an absolute FC of 1.5. In addition, we also investigated a more stringent cut-off of |log₂FC|>1. The overrepresentation of each set of candidate genes amongst biological pathways and other ontology sets was performed using g:Profiler. The gene-ontology molecular process sets overrepresented for the genes implicated in all three treatments were further subjected to clustering by semantic similarity via the REVIGO online platform.

We obtained genome-wide association study (GWAS) summary statistics of overall breast-cancer susceptibility, that is, all subtypes included (Zhang et al., Nature Genetics, 62:572-581)). Considering all cohorts included in the meta-analysis there were 133,384 cases and 113,789 controls of European ancestry. Non-palindromic common variants (frequency>1%) outside of the major histocompatibility complex were retained in order to construct a breast cancer polygenic risk score (PRS) and pharmagenic enrichment scores (PES) based on the targets of the FTO inhibitors. The breast cancer GWAS did not feature the UK Biobank cohort.

The breast cancer GWAS summary statistics were filtered such that only genes annotated to the target of FTO inhibition were retained, thus, forming the PES investigated henceforth in this study. Genic boundaries were extended 5kb upstream and 1.5kb downstream to capture regulatory variation. There were three different gene-sets considered as the FTO inhibitor target network: i) genes differentially expressed after both CS1 and CS2 treatment, ii) genes differentially expressed in all three treatments (CS1, CS2, and shFTO), and iii) genes differentially expressed in all three treatments using a stricter |log₂FC| cut-off of 1. After annotating variants to these three gene-sets, we performed clumping using the 1000 genomes phase 3 European reference panel (r²<0.1 per 250 kb clump) such that the remaining variants were in relative linkage equilibrium (LE). Three P-value thresholds were chosen to construct scores, and thus, each gene-set had three PES considered in the first instance: all SNPs mapped to the FTO inhibitor targets, nominally significant SNPs mapped to the FTO inhibitor targets (P_{GWAS}<0.05), and genome-wide significant SNPs mapped to the FTO inhibitor targets (P_{GWAS}<5×10⁻⁸). Specifically, a PES profile in individual i comprises sum of the effect size of j variants from the GWAS (\hat{β}ⱼ) annotated to at least one gene in set M, multiplied by the allelic dosage under an additive model (GᵢⱼϵG=0, 1, 2). PESᵢ=Σⱼ₌₁^{M}\hat{β}ⱼGᵢⱼ [1]. A genome wide PRS was constructed using the same three clumping and thresholding parameters, with the final scores averaged by the number of alleles in each participant. Scores were profiled using plink2.

The breast cancer FTO inhibition PES and PRS were profiled in the large, prospective UK Biobank (UKBB) cohort, which features extensive self-reported and primary care data related to cancer diagnoses. This research was conducted using the UKBB resource under the application 58432. The UKBB genotype data was previously processed such that unrelated individuals of white British ancestry were retained, along with other sample and variant level quality control considerations applied (Reay et al., 2021, medRxiv, https://doi.org/10.1101/2021.01.24.21250424). As a result, the composition of the full UKBB cohort in this study was 336,896 participants for which up to 13,568,914 autosomal variants were available (imputation INFO>0.8).

We defined prevalent breast cancer cases in the UKBB at time of analysis (July 2021) as females who satisfied at least one of the following criteria: i) self-reported breast cancer in their interview upon their visit/s to an assessment centre, ii) a relevant ICD-9 or ICD-10 code recorded via the linked national cancer registry inpatient data (ICD-9: 1740-1749, ICD-10: C500-C506, C508, C509). Controls were female-participants who did not self-report any cancer or had a relevant linked diagnosis recorded on the cancer registry at this same time-point. We tested the association of each breast cancer PES and PRS with prevalent breast cancer separately using a binomial logistic regression model covaried for age, 20 SNP derived principal components, and genotyping batch. The effect of additionally adjusting for PRS on the association between the PES and prevalent breast cancer was then quantified. Moreover, variance explained (Nagelkerke's R²) by each score was converted to the liability scale assuming a 3.6% population prevalence. The correlation between PES and PRS was also assessed, whilst residualised PES were derived to further model the PES/PRS relationship. These residualised scores are the scaled residuals from a linear model with the PES as the outcome variable and PRS, principal components, batch as the predictor, with the underlying aim to assess the extent that individuals with elevated PES are driven by the genome-wide polygenic signal (PRS) and/or technical factors related to population stratification and genotyping.

The results of this example are detailed henceforth.

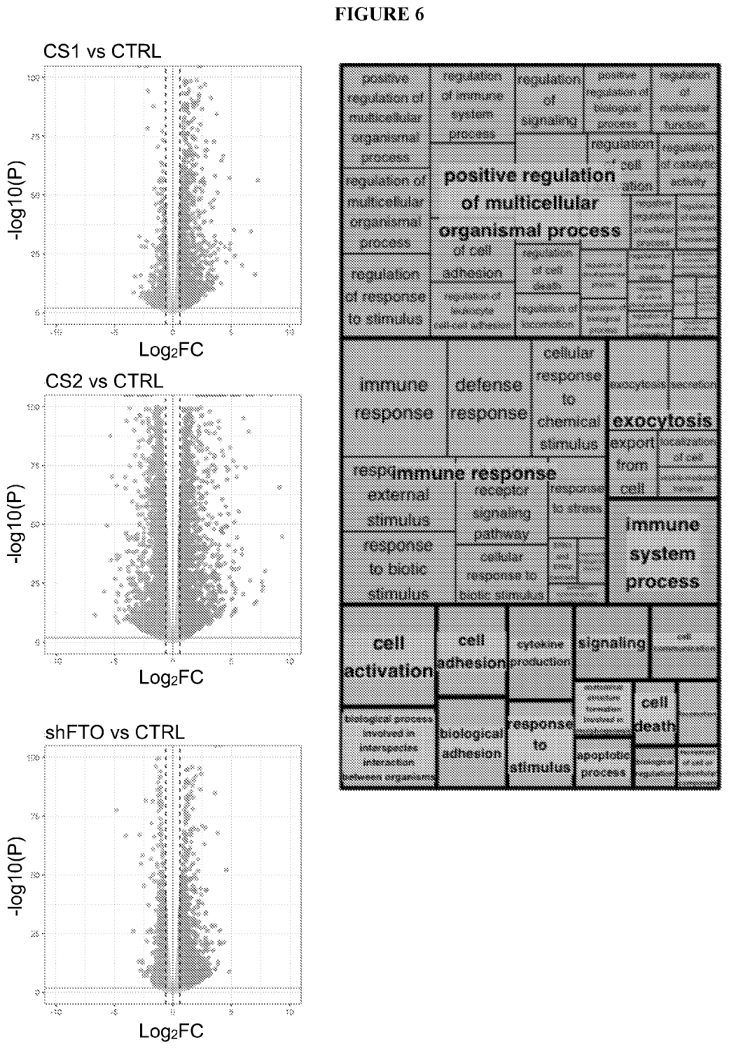

We investigated the mRNA correlates shared across in vitro treatment of the FTO inhibitors CS1 and CS2, as well as further comparing to the effect of FTO inhibition via a shRNA (shFTO, FIG. 6). Treatment with CS2 yielded more differentially expressed genes using our default threshold (N=5742, q<0.01 and |log₂FC|>0.6) than CS1 (N=2098), with 1543 genes implicated across both treatment conditions. We found that well characterised oncogenes were differentially expressed upon treatment with CS1 or CS2, such as BRCA2, MYC (c-MYC) and TERT. The overrepresented gene-sets for these 1543 genes largely represented immune related ontologies such as inflammatory response and cytokine binding, along with some pathways related to cell cycle control and apoptosis. Expression of the FTO gene itself as measured by mRNA expression was not significantly altered by either inhibitor, which is likely reflective of that mode of action of these drugs on the FTO protein. Notably, BRCA2, one of the most well-characterised breast cancer risk genes, was significantly upregulated by both compounds. We then further intersected the CS1 and CS2 differentially expressed genes with those correlated with shFTO treatment and found 597 genes impacted across all three conditions. Clustering of gene ontology (GO) molecular process sets by semantic similarity was also consistent with a strong immune component of these shared genes (FIG. 6D). A more stringent fold change threshold of |log₂FC|>1 further reduced this list down to 225 genes, and thus, represents the most strongly correlated genes with all three treatments. The overrepresentation analyses also supported strong enrichment for these genes amongst immune related processes, consistent with the hypothesis that FTO inhibition may help to overcome immune evasion.

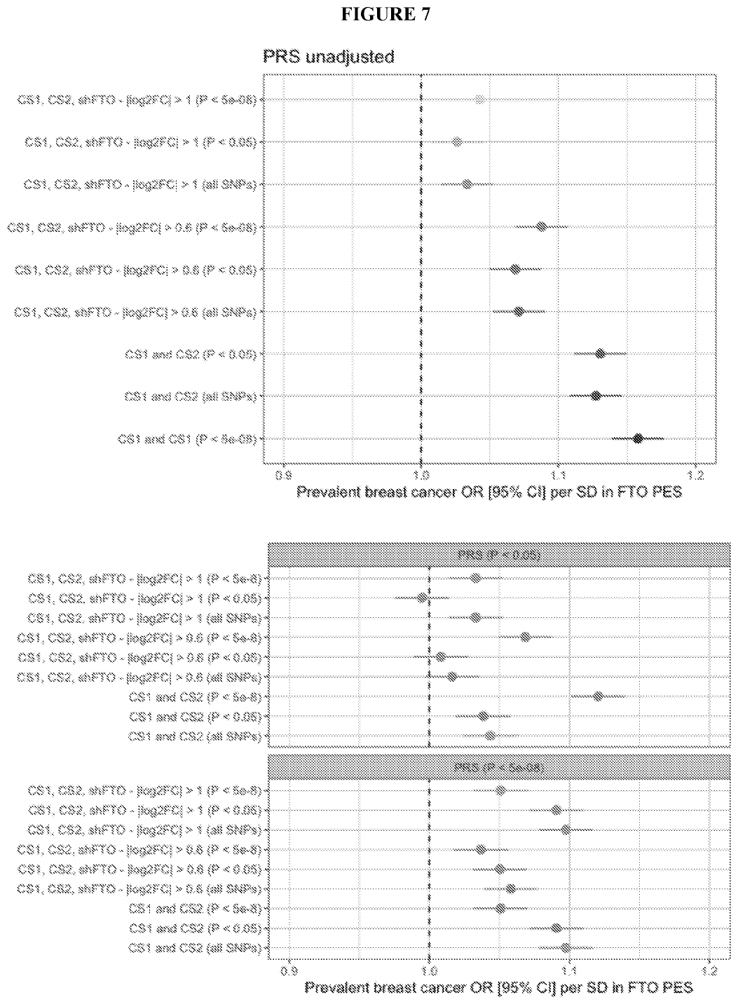

The association of pharmagenic enrichment scores (PES), constructed from the three-sets of FTO inhibition related genes, was tested for prevalent breast cancer amongst female participants in the UK Biobank cohort (FIG. 7). After performing variant and participant level quality control, there were 11635 prevalent cases of breast cancer and 142291 female participants with no history of any cancer at time of censoring that acted as controls. Prevalent breast cancer cases were significantly older (approximately three years mean difference), in line with expectation. All three versions of the FTO target PES were enriched amongst women ever diagnosed breast cancer, that is, the PES constructed from genes differentially expressed after CS1 and CS2 treatment and all three treatments with a |log₂FC| threshold of 0.6 or 1, respectively. This enrichment was seen regardless of whether scores were constructed from all SNPs mapped to the gene-set, nominally significant SNPs only, or genome-wide significant SNPs only. The CS1 and CS2 shared genes PES demonstrated the largest effect sizes. Specifically, this PES constructed using 28 independent genome-wide significant SNPs mapped to those FTO target genes explained approximately 0.42% of the variance in prevalent breast cancer on the liability scale, with each standard deviation (SD) increase in the PES associated with a 15.81% [95% CI: 130.92%, 170.69%] increase in the odds of breast cancer (P=1.54×10⁻⁵²). The same PES but using a nominal significance threshold to select SNPs, 2845 independent SNPs, obtained a similar effect size: OR per SD=1.13 [95% CI: 1.11, 1.15], P=1.62×10⁻³⁶, R²=0.29%. A genome-wide PRS for breast cancer was associated with a greater increase in the odds of the disease, as would be expected, with the genome-wide significant SNP PRS the best performing, explaining approximately 3.63% of the variance on the liability scale. Crucially, whilst the effect size of the CS1 and CS2 shared genes PES was marginally reduced when adjusting for the effect of PRS, the PES remained highly significant—for example, for the genome-wide significant SNP FTO PES after conservatively covarying for PRS: OR per SD=1.05 [95% CI: 1.03, 1.07], P=6.11×10⁻⁷ (χ² test of residual deviance). In other words, the FTO related PES were independently associated with prevalent breast cancer and were not likely to be purely a product of non-specific inflation of PRS.

The above embodiments in this example represent a mechanism by which a directional anchor PES for a compound that inhibits FTO could utilised in the precision treatment of breast cancer. In this embodiment, patients with high FTO associated PES may be expected to display a stronger response to an FTO inhibitor. Accordingly, these scores were significantly elevated in breast cancer cases even after adjustment for the background of elevated genome wide genetic risk among female participants in the UK Biobank cohort.

1. A method for treating a complex disorder in a human subject comprising:
a. identifying one or more directional anchors which encompass one of the following:
i. selecting a therapeutic agent or agents for the treatment of a complex disorder;
ii. selecting a therapeutic target, as embodied by a therapeutically targetable entity, for the treatment of a complex disorder;
b. obtaining a set of genes or other biological factors related to the directional anchor identified in step (a), comprising the steps of:
i. identifying genes predicted to interact with the known targets of a therapeutic agent identified in step (a)(i), including, but not limited to, as derived from physical interaction databases, expression studies; curated databases and the published medical literature;
ii. in the case of a therapeutic target directional anchor identified in step (a)(ii), identify genes predicted to interact with that target including, but not limited to, as derived from physical interaction databases, expression studies; curated databases and the published medical literature;
c. identifying individuals who may be more sensitive to the selected treatment through the following;
i. obtaining data representing genome-wide variants from a plurality of individuals with the complex disorder and a plurality of individuals without the complex disorder;
ii. obtaining estimated effect sizes on the disorder from said plurality of variants;
iii. generating a plurality of annotations corresponding to the genes identified in either step (b)(i) or step (b)(ii);
iv. identifying genome-wide variants that intersect the annotations from step (c)(iii);
v. obtaining data representing genome-wide variants in a biological sample from a subject;
vi. calculating a predictive polygenic score for only variants annotated to the pharmacologically-relevant gene set related to the directional anchor identified in step (a) from the subject's genome wide variant data
vii. identifying individuals more sensitive to the treatment identified in step (a) from the predictive polygenic score from step (c)(vi) that is elevated relative to a reference value indicates that the subject may be sensitive to the at least one annotated therapeutic agent,
d. treating the subject with the selected therapeutic agent.

2. The method of claim 1, wherein the data representing genome-wide variants is selected from the group consisting of single nucleotide polymorphism (SNP) genotype data, copy number variant (CNV) data, gene deletion data, gene inversion data, gene duplication data, gene fusion data, variable number tandem repeat data, microsatellite repeat data, trinucleotide repeat expansion data, splice variant data, haplotype data, or combinations thereof.

3. The method of claim 1, wherein the data representing genome-wide variants is genome wide association study (GWAS) summary statistics.

4. The method of claim 1, wherein the variants are selected from the group consisting of common SNPs, CNV, translocations, gene deletions, gene inversions, gene duplications, gene fusions, variable number tandem repeats, microsatellite repeats, trinucleotide repeat expansion data, splice variants and haplotypes associated with the complex disorder.

5. The method of claim 1, wherein the complex disorder is schizophrenia.

6. The method of claim 1, wherein the complex disorder is bipolar disorder.

7. The method of claim 1, wherein the complex disorder is breast cancer.

8. A computer-based genomic annotation system comprising non-transitory memory configured to store instructions and at least one processor coupled with the memory, the processor configured to:
a. receive genome-wide variant data from a plurality of individuals with a complex disorder and a plurality of individuals without a complex disorder;
b. specify a directional anchor as comprised by a therapeutic agent or therapeutic target;
c. identify genes biologically related to the directional anchor from step (b), whereby this would include, but is not limited to, processing data from physical interaction databases, expression studies; curated databases and the literature
d. for each of the identified gene sets, generating a plurality of annotations corresponding to a plurality of predefined annotation categories, wherein the plurality of predefined annotation categories comprise a biological pathways annotation category, a drug target annotation category and an approved therapeutic agent annotation category; and

9. The computer-based genomic annotation system of claim 8, further comprising a separate computational process to interrogate the genotype of an individual subject to select an agent suitable for the treatment of a complex disorder comprising:
a. receiving genome-wide variant data from a biological sample from the subject;
b. calculating a predictive polygenic score for the pharmacologically-relevant gene set related to the directional anchor from the subject's genome wide variants data; and
c. selecting at least one therapeutic agent from the treatment of the complex disorder as comprised by the directional anchor, wherein a predictive polygenic score calculated that is elevated relative to a reference value indicates that the subject may be sensitive to the at least one annotated therapeutic agent.
